(19) United States
(12) Patent Application Publication
(10) Pub. No.: US 2022/0218754 A1
(43) Pub. Date: Jul. 14, 2022

(54) A METHOD TO MANUFACTURE A COMPOSITION COMPRISING PLATELET-RICH PLASMA, AN APPARATUS FOR CENTRIFUGATION AND A KIT UTILISABLE FOR ACTUATING THE METHOD, THE COMPOSITION, AND USE OF THE COMPOSITION

(71) Applicant: PROMETHEUS S.r.l., PARMA (IT)

(72) Inventors: Riccardo DELLA RAGIONE, MIRANDOLA (MO) (IT); Valentina MENOZZI, ALTIDONA (FM) (IT); Alice MICHELANGELI, MIRANDOLA (MO) (IT)

(21) Appl. No.: 17/609,687

(22) PCT Filed: May 13, 2020
(86) PCT No.: PCT/IB2020/054534

§ 371 Date: Nov. 8, 2021

(30) Foreign Application Priority Data
May 15, 2019 (IT) 102019000006867

(51) Int. Cl. A61K 35/19; B01D 21/26; A61K 9/06; A61K 47/34; A61K 47/36

(52) U.S. Cl. CPC A61K 35/19; B01D 21/262; B01D 2221/10; A61K 47/34; A61K 47/36; A61K 9/06

(57) ABSTRACT

A plasma-rich platelet composition is in a gel form and includes a polymeric scaffold and a polymeric sponge. The scaffold is made of polylactic acid and has a grid structure with a thickness between 5 and 500 μm. The polymeric sponge is manufactured with a material selected from the group consisting of alginate, gelatin, collagen and chitosan and combinations thereof and has a thickness between 1 mm and 10 mm. A container holding the composition has a deformable wall, a filter and a connector for conveying gelling fluid.

### DESCRIPTION OF THE INVENTION

### Field of the Invention

The present invention relates to a method, an apparatus for centrifugation and a device to manufacture a composition comprising platelet-rich plasma, a kit for the device, the composition itself and a use of the composition.

The present invention has, in particular, an advantageous application to manufacture a composition comprising platelet-rich plasma obtained from blood collected from a human or animal patient.

### Description of the Prior Art

For some time the use of compositions has been known comprising blood derivatives, such as for example platelet-rich plasma (commonly called “PRP”) for the treatment of skin lesions and osteochondral or joint pathologies. The PRP is obtained starting from whole blood collected from a patient who might be human or animal, by means of a process of single or double centrifugation. Double centrifugation enables carrying out the separation of the components of whole blood substantially in two steps.

In the first step there is a separation of the buffy coat (comprising the majority of the white blood cells) and the red blood cells from the plasma containing the platelets.

The second step includes the depositing of the platelets in the form of a pellet on the bottom of the container and, in the upper portion, the platelet-free or platelet-poor plasma.

To obtain the final composition comprising PRP, the pellet of platelets formed is resuspended and solubilised in a volume of plasma that is smaller than the starting volume, so as to concentrate the platelets. The liquid composition comprising PRP has a concentration of platelets at least 4-6 times greater than the initial concentration, keeping the platelets vital, active and functional, able to release growth factors.

In the devices of known type, however, there is control and command exclusively of the acceleration and the time of acceleration. The deceleration, on the other hand, follows the natural loss of velocity of the particles due to inertia and friction. Therefore, as the random deceleration can be too long or too short, with a negative influence on the quality of the PRP obtained, due to the stress to which the platelets are subjected.

Further, the known devices that carry out a centrifugation of a vertical type create an internal vortex during the halting of the acceleration which causes remixing between red blood cells and plasma with a consequent greater quantity of red blood cells in the final composition, which has the further disadvantage of stimulating an excessive immune system mediated response.

The known devices are able to automatically manufacture only the composition in liquid shape comprising PRP. The known devices are however unable to automatically manufacture, in a sterile and controlled environment, the composition comprising PRP in gel form. In fact, the operator must manually add a gelling agent to the liquid composition of PRP (such as for example Calcium Gluconate, Calcium Chloride and/or thrombin) and thereafter must incubate the composition at a temperature of about 37° C., up to complete gelling.

Therefore, to date, the operation of manufacturing the composition (comprising PRP) in gel form has a plurality of drawbacks. The manual process of manufacturing the composition in gel form does not give a high level of safety due to human errors that might intervene. As it is not possible to predispose a movable fume hood to work in sterile conditions in any station, it is clear that the composition in gel form obtained cannot always satisfy the requisite of sterility, as it is not possible to have a fume hood in every station. Further, as the process is purely manual, there is a component of uncertainty which does not guarantee good process repeatability, i.e. it is not possible to guarantee the same quality of the composition comprising PRP which has been manufactured by two production processes that are successive and distinct. The composition in gel form of known type is further poorly uniform, for example in terms of PRP distribution internally thereof. With the know method, the composition in gel form obtained is not completely gelled, having a high amount of component that is still liquid or semi-solid, which tends to drip, losing the active ingredients that characterise it and foul the surrounding environment. Further, the gel composition of known type does not have a stable shape and therefore deforms, making its handling and application complicated.

Further, the known composition has: a limited percentage of platelets retrieved by the whole blood which are preserved in the final product and a low factor of concentration (i.e. the ratio between the platelet contents with respect to the starting product).

Consequently, the known gel composition has poor quality from the point of view of functionality, effectiveness and applicability of the composition itself.

Daniel Tzu-BiShih et al. “Preparation, quality criteria, and properties of human blood platelet lysate supplements for ex vivo stem cell expansion” New Biotechnology” Volume 32, Issue 1, 25 Jan. 2015, Pages 199-211 describes a PRP obtained via two centrifugation steps. The first step of centrifugation of the whole blood, to which an anticoagulant has been added, is carried out at 1000 g for 10 minutes at t 20-22° C. The second centrifugation of the resulting supernatant is then carried out at about 3000× g for 5 minutes at 21-22° C. and the deposited concentrate is re-suspended in 50-70 mL of plasma.

### SUMMARY OF THE INVENTION

An object of the present invention is therefore to provide a method, an apparatus for centrifugation and a device to manufacture a composition, in particular comprising platelet-rich plasma, a kit for the device, the composition itself and a use of the composition which are free of the drawbacks of the prior art, which are easy and economical and which are of high quality. Note that by joining the apparatus for centrifugation and the single-use kit the device is advantageously obtained where there is no need to wash and/or sterilise the parts that have come into contact with the blood, the red blood cells, the plasma, the white blood cells, the platelets and the PRP.

The present invention provides to a method, a centrifuging apparatus and a device to manufacture a composition, in particular comprising platelet-rich plasma, a kit for the device, the composition itself and a use of the composition according to the contents of the appended claims.

### BRIEF DESCRIPTION OF THE DRAWINGS

For a better understanding of the invention, two embodiments are illustrated, purely by way of non-limiting example, in which:

FIG. 1 is a perspective and schematic view, with several parts removed in order to better highlight others, of the apparatus for centrifugation of the invention comprised in a device to manufacture a composition comprising platelet-rich plasma, in accordance with the present invention;

FIG. 2A is a diagram of an embodiment of the kit of the invention;

FIG. 2 is a fluid-dynamic diagram of the device of FIG. 1;

FIG. 3A illustrates a first embodiment of a collection unit for the liquid composition; and

FIG. 3B illustrates a second embodiment of the collection unit for the composition in gel form.

### DESCRIPTION OF THE PREFERRED EMBODIMENTS OF THE INVENTION

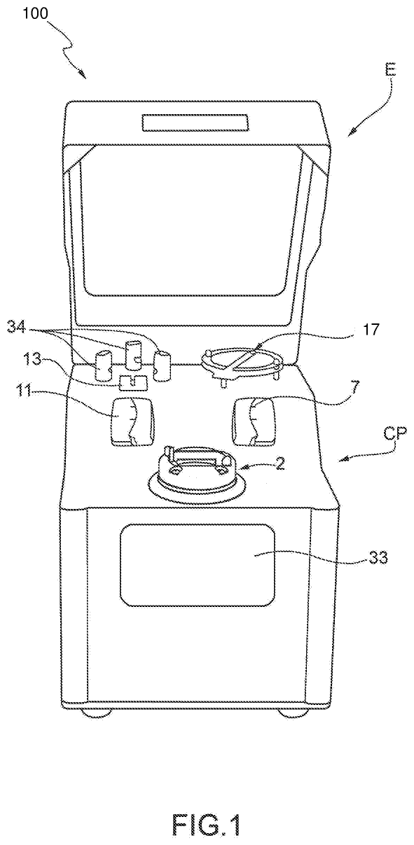

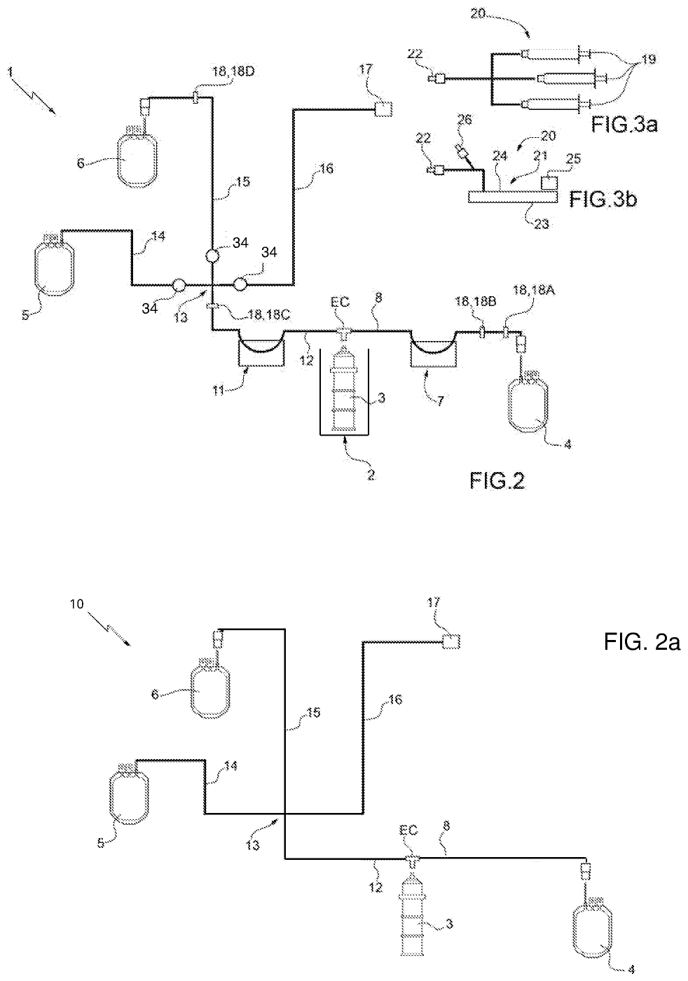

In FIG. 1 the reference numeral (100) denotes in its entirety an apparatus for centrifugation according to the invention, while in FIG. 2a) reference number (10) denotes a kit according to the invention. In FIG. 2, reference numeral (1) denotes in its entirety a device to manufacture a composition comprising (in particular constituted by) platelet-rich plasma (which in the following will be denoted as PRP).

The composition can advantageously be injectable (i.e. in the form of liquid) or applicable and/or suturable on the skin lesion (i.e. in gel form, in particular a gel plaster).

The PRP is obtained from whole blood collected from a human or animal patient which is subsequently centrifuged, i.e. separated. The quantity of whole blood collected is preferably comprised between 10 and 200 mL.

The centrifuging apparatus (100) comprised in the device (1) illustrated in FIG. 1 comprises a main body (CP) which substantially houses the units of the device (1) and a closing element (E) hinged to the main body (CP).

The closing element (E) is configured to be arranged between an open position (FIG. 1), in which the operator can access the units, and a closed position (not illustrated), in which the operator cannot access the units of the device (1). The device (1) advantageously comprises elements (not illustrated) for verifying the correct closing of the closing element (E). Note that FIG. 1 also includes numerical references (13) and (17) indicating the position in which the following elements comprised in the kit according to the invention will be respectively positioned:—a node (13) constituted by a four-way connector (13), and a collection interface (17). The device (1), obtained by advantageously combining the kit (10) and the apparatus (100) for centrifugation, comprises a centrifugation unit (2), constituted by the separation container present in the kit and the centrifugation station of the apparatus (100) for centrifugation, and configured to separate the components of the whole blood as a function of the different density and the physical state thereof (liquid or solid), by exploiting the action of the centrifugal force. The centrifugation unit (2) is provided with a separation container (3) (illustrated in FIG. 2) which is set in rotation about an axis thereof (substantially vertical) by a drive unit, such as for example an electric motor. In fact, the apparatus (100) for centrifugation is preferably a single-station centrifuge configured to rotate the centrifugation container (3), when housed in the relative centrifugation station, about the relative vertical axis.

The centrifugation unit (2) advantageously carries out a double centrifugation of the whole blood, so as to separate the blood into the single components. The separation container (3) is therefore set in rotation during two successive centrifugation cycles in order to separate the components of the whole blood so as to obtain the platelet-rich plasma (PRP).

During the first centrifugation the device (1) separates the whole blood into plasma comprising platelets, buffy coat comprising white blood cells and red blood cells. The buffy coat and the red blood cells are waste materials and are subsequently removed. In the second centrifugation the plasma is separated into platelets; a pellet of platelets and platelet-free or platelet-poor plasma, is formed. Lastly, substantially the whole volume of pellet of platelets deposited on the bottom of the separation container (3) is resuspended in a part of the plasma, in order to obtain the PRP. The part of the plasma in which the resuspension of the pellet of platelets takes place corresponds to about 10% of the initial quantity (preferably in volume) of the whole blood. The remaining part of plasma (about 90% of the initial quantity) is eliminated.

In a possible embodiment, not illustrated, the separation container (3) comprises only a lateral wall having a substantially cylindrical shape. The separation container (3) advantageously comprises a relative inlet, a relative fixed wall and a movable wall which define a relative variable internal volume for containing a liquid, wherein the movable wall is movable with respect to the fixed wall to vary the internal volume. This enables the container to vary the volume thereof in the device (1), following the activation of the pump (7 or 11). The separation container (3) is connected at the upper end thereof to the centrifugation unit (2) via a connecting element (EC). The connecting element (EC) prevents the transmission of vibrations to the device (1). The connecting element superiorly closes the separation container (3) and in a preferred embodiment of the invention prevents the transmission of motion to the other components of the device (1). In fact, the connecting element (EC) comprises: a three-way connector, wherein each relative different single way of the connector is hydraulically connectable, respectively to the first end of the second conduit (12), to the second end of the first conduit (8) and to the inlet of the separation container (3); and, optionally, means for reducing friction, preferably constituted by a rotary joint, arranged at one of the ways to enable hydraulic connection of the way and to the inlet of the separation container (3) even when the separation container is subjected to centrifugation. If the means for reducing friction are present, the connecting element (EC) can remain hooked to the container (3) even when the container is in the centrifugation step and will not transmit the motion to the conduits (8 and 12) to which it is connected. If the means for reducing friction are not present, the connecting element (EC) must be detached from the centrifugation container (3) during the centrifugation step.

According to this embodiment, the separation container (3) does not have a bottom wall (it is inferiorly open). A piston is arranged internally of the separation container (3), configured to slide internally thereof with an alternating motion. The piston is arranged in such a way as to inferiorly close the separation container (3). The piston can have a seal which inferiorly and sealedly closes the separation container (3).

As illustrated in FIG. 2, the device (1) comprises three containers 4, 5, and 6.

Each container (4, 5 and 6) is advantageously manufactured for example by a bag for medical infusions.

The containers (4, 5, 6) can advantageously be arranged in housings made in the main body (CP) or can be hung on appropriate hooks at the lateral walls of the main body of the device (1). The collection container (4) is filled with collected whole blood, to which an anticoagulant (in particular, ACD-A) has been added. The container (5) for intermediate storage is initially emptied and functions substantially as an intermediate store for a component (in particular for the plasma comprising platelets or the platelet-free or platelet-poor plasma). The container (6) is filled with a cleaning liquid.

The whole blood housed in the container (4) is supplied to the centrifugation unit (2) by means of a pump (7). The pump (7) is arranged along a conduit (8), which connects the collection container (4) with the centrifugation unit (2).

The pump (7) is advantageously peristaltic and the conduit (8) passes through the pump (7). The first centrifugation preferably takes place with an acceleration having an acceleration value (A1) and/or a preset or presettable acceleration duration (TA1). The acceleration value (A1) is advantageously comprised between 100 and 2500 g (where g indicates gravitational acceleration). The duration of acceleration (TA1) is comprised between 1 and 20 min. Following the acceleration a controlled deceleration takes place which has a preset or presettable deceleration value (D1) and/or a duration (TD1), in such a way as to realise the separation of the single components of the whole blood.

The term controlled deceleration is meant as a piloted deceleration, i.e. with a preset deceleration value (D1) (i.e. with the deceleration value to be dissipated—rad/min² or rad/s²) and/or a deceleration duration value (TD1) between which the blood particles must be substantially still. The term controlled deceleration does not therefore relate to the natural deceleration due to inertia and friction, which takes place downstream of the switching off and therefore of the separation container (3).

The deceleration value (D1) is correlated to the acceleration value (A1). The higher the acceleration value (A1), the more gradually (i.e. slowly) the deceleration must take place, so as to reduce as much as possible the formation of vortices in the blood during the slowing.

The deceleration value (D1) is advantageously comprised between: 0.2 and 0.5 rad/min². The deceleration duration (TD1), on the other hand, is comprised between 2 and 20 min. In this case, the deceleration value (D1) is preferably comprised between 0.0009 and 0.5 rad/sec², and advantageously it is 0.2 and 0.5 rad/sec².

At the end of the piloted deceleration of the first centrifugation step, the centrifuged blood is left to rest (i.e. brought into stasis) for a time required to facilitate the precipitation of the red blood cells in the separation container (3). This time is typically about 2 min.

At the end of the first centrifugation, the plasma containing the platelets is arranged on the upper portion of the separation container (3). The red blood cells separated from the whole blood are arranged on the lower portion, i.e. on the bottom, of the separation container (3) and the buffy coat (comprising the majority of the white blood cells) is arranged between the plasma and the red blood cells. The plasma comprising platelets is supplied to the container 5 configured for the intermediate storage thereof. The supply from the centrifugation unit (2) to the container (5) is done by means of a pump (11). The pump (11) is arranged along a conduit (12) which subsequently divides, at a node (13), into the conduits (14, 15 and 16). The node (13) is substantially constituted by a multi-way connector, in particular a 4-way connector. The conduit (14) connects the node (13) to the container (5). The pump (11) is advantageously peristaltic. This enables not having to clean and sterilise the peristaltic pumps (7 and 11) after each use.

The conduit (15) connects the node (13) to the container (6).

The conduit (16) connects the node (13) to a collection interface (17).

When the pump (11) has conveyed the plasma comprising platelets to the container (5), the separation container (3) (empty, or rather comprising red and white blood cells) is subjected to a cleaning cycle to eliminate any residues in it. In particular, the pump (11) supplies, and then collects, the cleaning liquid from the container (6) towards and from the separation container (3) via the conduit (15). In a further embodiment of the method the pump (11) takes the cleaning liquid from the container (6) to the recipient (3), and the pump (7), via the conduit (8), collects and directs the cleaning liquid, from the recipient (3) towards the container (4).

After the cleaning cycle the plasma comprising platelets is newly supplied by means of the pump (11) towards the centrifugation unit (2), in order to be subjected to a second centrifugation. The second centrifugation takes place with an acceleration having an acceleration value (A2) and/or a duration (TA2) that are preset or presettable.

The acceleration value (A2) is advantageously comprised between 100 and 2500 g. The duration of acceleration (TA2) is comprised between 1 and 20 min.

Following a second acceleration a second controlled deceleration takes place and/or a preset or presettable deceleration value (D2) and/or a duration (TD2), so as to separate the pellet of platelets from the platelet-poor, or platelet-free plasma. The pellet of platelets deposits on the bottom and sides of the separation container (3); while the plasma without or with only a negligible quantity of platelets becomes arranged in the upper part of the separation container (3).

The acceleration value (A2) of the second centrifugation is advantageously greater than the acceleration value (A1) of the first centrifugation. On the other hand, the controlled deceleration of the second centrifugation (TD2) is instead lower than the controlled deceleration duration (TD1) of the first centrifugation.

A part of the volume of the platelet-free or platelet-poor plasma (for example about 90% of the initial quantity—in volume—of the whole blood) is rejected and fed into the container (5). The remaining volume of plasma (about 10% of the initial quantity—in volume—of the whole blood) is used to re-solubilise at least a part of pellet of platelets, preferably substantially the whole pellet of platelets. The resuspension of the platelets in at least a part of plasma takes place by activating the centrifugation unit (2) with brief centrifugations with low acceleration, for example in the order of 20 g repeated for example from 5 to 10 times, so as to obtain the PRP.

The composition comprising PRP can then be collected from the collection interface (17).

The device (1) advantageously comprises a electronic control unit (ECU). The electronic control unit (ECU) is configured to activate the centrifugation unit (2) with the acceleration value (A1 or A2) and/or for a duration of acceleration (TA1 or TA2) and/or with a preset deceleration value (D1 or D2) and/or a preset or presettable deceleration duration (TD1, TD2), in such a way as to realise the separation of the whole blood or to realise the suspension of the platelets in at least a part of plasma.

As illustrated in FIG. 2, the device (1) comprises sensors (18) configured to detect at least a characteristic from among: the infeed flow rate, the correct insertion of the conduits (8, 12 and 15), any presence of air bubbles, the presence of the fluid to be supplied in the relative conduit (8, 12, 15) and/or the change in turbidity of the fluid to be supplied.

The sensors (18) comprise for example liquid presence sensors or sensors of another type. The sensors (18) can be different to one another.

The sensors (18) are preferably four in number, i.e. sensors (18A, 18B, 18C and 18D). The sensors (18A and 18B) are arranged at the conduit (8) downstream of the collection container (4) and are arranged in succession one after the other. The sensors (18A and 18B) enable detection of the emptying of the container (4) and the presence of any air bubbles present in the blood flow.

The sensor (18C) is arranged along the conduit 12 and upstream of the node (13). The sensor (18C) detects the turbidity of the fluid that the pump (11) is transporting, thus differentiating between plasma (typically yellow in colour) and red blood cells (typically red in colour). The sensor (18C) enables interruption of the flow towards the container (5), at the moment when the red blood cells are detected. Therefore the sensor (18C) enables only the plasma (with or without platelets) to be temporarily stored in the container (5).

The sensor (18D) is instead arranged along the conduit (15) and upstream of the container (6) and detects the cleaning fluid infeed flow rate.

In addition, the sensors (18A and 18B or 18C and 18D) enable calibrating respectively pump 7 or 11. The calibration consists of a correction of the effective flow rate (i.e. step/mL) that the pump (7 or 11) really delivers. As the effective flow rate of the pump (7 or 11) is strongly dependent on the wear on the springs internally of the pump, on the hardness of the conduit inserted in the pump (7 or 11), on the opening or closing of the pump (7 or 11), etc., the effective flow rate will have to be recalculated at each start-up of the device (1). In particular, as the length of the portion of conduit (8) comprised between the sensors (18A and 18B or 18C and 18D) remains constant (i.e. the length of the single conduits comprised between the two sensors does not vary), the volume internally of the conduits in this portion is always constant and known. Therefore, by determining the number of steps which the pump (7) or (11) carries out to move the volume between the sensor (18A and 18B) or (18C or 18D), at each start-up of the device (1), the effective flow rate of the pump (7 or 11) can be recalculated.

In a possible embodiment, not illustrated, the liquid composition comprising PRP could be collected directly from the collection interface (17).

In alternative embodiments illustrated respectively in FIGS. 3A and 3B the device (1) also comprises a collecting unit (20) of the composition connected or connectable to the collection interface (17) of the device (1). In particular, the collecting unit (20) is connectable to the device (1) via a connecting means (22), in particular a Luer connector.

According to the embodiment illustrated in FIG. 3A, in the case of the liquid composition, the device (1) does not have to carry out a further operation with respect to what has been described up to this point. Therefore, in this case, the liquid composition is ready to be collected by at least a collecting container (19), preferably a syringe, of the collecting unit (20). The collection unit advantageously comprises more than one container (19), for example three.

According to the embodiment illustrated in FIG. 3B, in the case of composition in gel form, the collecting unit (20) comprises a gelation container (21). The gelation container (21) is preferably made of PVC. The solidification of the composition comprising PRP is done in the gelation container (21). In this case, the collection interface (17) of the PRP is configured to be connected to the gelation container (21) via a connecting means (22), in particular a Luer connector, arranged on the gelation container (21). The gelation container (21) has a connecting means (26), in particular needle-less, for connecting a conveying means of a gelling fluid and can, preferably, also have an air filter (25). The air filter (25) (if present) enables the air to exit and further enables the sterility of the gelation container (21) to be maintained. The air filter (25) is in particular hydrophobic. The gelling fluid comprises (in particular is constituted by) calcium gluconate. The conveying means of the gelling fluid is preferably a syringe, but might also be supplied automatically from the device (1).

According to the embodiment illustrated in FIG. 3B, the connecting means (26) is arranged at the conduit (16).

The gelation container (21) comprises at least a lower wall (23) and/or an upper wall (24). At least the lower wall (23) and/or the upper wall (24) is elastically deformable, so as to deform under the action of a compression force exerted thereon.

A polymeric scaffold is arranged in the gelation container (21), in particular manufactured with a biopolymer such as for example polylactic acid. The polymeric scaffold functions as a support matrix for the composition comprising PRP in gel form, maintaining the flexibility thereof.

Therefore the scaffold contributes to realising the composition in gel form, in particular a plaster that is applicable and suturable.

The scaffold advantageously has a grid structure, with a thickness comprised between 5 and 500 μm, preferably comprised between 100 and 350 μm. The grid structure is obtained, for example, by superposing two layers of material.

In a possible embodiment, not illustrated, the polymeric scaffold can be manufactured by 3D printing.

In a possible embodiment, a polymeric sponge can be arranged in contact with the polymeric scaffold. The polymeric sponge is made for example of a material selected from among: alginate, chitosan, gelatin and/or collagen. The polymeric sponge has a thickness comprised between 1 mm and 10 mm, preferably between 1 mm and 3 mm. The polymeric sponge (if present) enables increasing the flexibility of the composition in gel form and accelerate the gelling process, preventing loss of material.

In a possible alternative embodiment, in order to improve the handling and removal of the composition in gel form, the gelation container (21) can be provided with a facilitated opening element so as not to damage the composition.

In a possible embodiment, not illustrated, the device (1) comprises a housing for the gelation container (21). The housing comprises a plate and a support (not illustrated) which are connected to one another.

The plate and support are advantageously connected to one another preferably by means of the elastic return means (not illustrated) so as to exert a compression force on the gelation container (21) interposed there-between.

The gelation container (21) is compressed during the insertion in the housing, so that the air is made to exit completely. Th elastic return means are advantageously hinges or springs. The elastic return means are preferably at least one, preferably at least four. The elastic return means (if present) enable compressing the gelation container (21) proportionally to the quantity of composition comprising PRP which is introduced into the gelation container (21) and further guarantee a greater expulsion of the air present in the gelation container (21)

The housing is preferably provided with a heating unit for heating, in use, at least a wall (23) or (24) of the gelation container (21) at least at an incubating temperature (TI) comprised between 35° and 42°, in particular about 37°, to strengthen the PRP in the gelation container (21) The heating unit is for example a heating electrical resistance.

The housing of the gelation container (21) is advantageously made of aluminium in order to have a homogeneous incubating temperature (TI). In this way, all of the gelation container (21) is heated substantially to the same incubating temperature (TI).

According to what is illustrated in FIG. 1, the apparatus (100) also comprises an interface unit (33) which comprises a screen. Therefore, the device (1) also comprises an interface unit (33) which comprises a screen. The screen can be for example a touch screen, so as to enable viewing the output data and enter the input data. The input data comprises, for example, the choice of program to run on the device (1).

In the preferred embodiment the device (1) comprises at least a tube-clamp valve (34). In particular, the device 1 comprises three tube-clamp valves (34). The tube-clamp valves (34) are arranged downstream of the node (13) respectively at conduits (14, 15 and 16).

In use, the operator arranges the containers (4, 5 and 6) in appropriate housings and carries out the various steps indicated on the interface unit (33) (if present), selecting the program to run on the device (1).

Experimental tests have shown that, for example, in the case of whole blood collected from a horse, there are advantageous results in carrying out the two centrifugations with the following values:
the first centrifugation with the acceleration value (A1) about 200 g, the acceleration duration (TA1) of about 3 minutes and the controlled deceleration duration (TD1) of about 14 minutes;
the second centrifugation with the acceleration value (A2) about 750 g, the acceleration duration (TA2) of about 5 minutes and the controlled deceleration duration (TD2) of about 5 minutes.

Experimental tests have also shown that the time for completing gelling, in the case of PRP obtained from horse blood, is comprised between 10 and 40 minutes, in particular of the order of 20 minutes. Further, the experimental tests have shown that the overall duration of the process to manufacture the composition in gel form is comprised between 30 and 90 minutes, and in particular is 60 minutes.

Comparative tests among compositions manufactured with the above-mention device (1) and compositions manufactured with devices of known type have shown that the percentage of retrieved platelets and the factor of concentration (ratio between the quantity of platelets present in the whole blood and quantity of platelets in the final preparation) obtained with the device (1) and the relative protocols, are greater than those of the known-type devices, thus enabling greater factors of concentration to be obtained, without significantly reducing the volume of plasma containing platelets.

The present invention also relates to a single-use sterile kit to manufacture the injectable composition (in the following termed “injectable single-use kit”) and the single-use sterile kit to manufacture the injectable composition in gel form (in the following termed “gel single-use kit”) to be used with the apparatus (100) for obtaining the device (1).

The above-described kits comprise at least an element selected from among: a bag for collecting the blood (i.e. the collection container (4)); a dose of anticoagulant fluid; a needle and a tube for the collection; a 250 mL washing bag (i.e. the container 6) containing the cleaning liquid, such as for example saline; an 250 mL empty bag that functions as an intermediate store for temporary storage of the plasma (i.e. the container (5) for intermediate storage); the conduits (8, 12, 14, 15 and 16) which are preferably at least partly made of PVC; a multi-way connector, in particular a 4-way connector (i.e. the node (13)); and the separation container (3) provided, for example, with the connecting element, the piston and the seal for the piston.

Apart from the components indicated in the foregoing, the “injectable single-use kit” further comprises, at least a syringe for intra-articular or subcutaneous injection of the liquid composition comprising PRP.

The “gel single-use kit” comprises, as well as the above-indicated components a gelling fluid syringe, the gelation container (21), the polymeric scaffold and preferably a polymeric sponge. The composition comprising PRP obtained with the device (1) and/or the method described up to this point can be used as a medication, in particular for the treatment of skin lesions and/or of osteochondral or joint pathologies.

The above-described composition can be used for the preparation of a medication for the treatment of skin lesions and/or of osteochondral or joint pathologies.

The invention described up to this point has a plurality of advantages.

Primarily, the device (1) has the advantage of being versatile, as the same device (1) can be used to realise various formulations of PRP.

Further, simply by varying the collecting unit (20) it is possible to obtain two compositions having two different consistencies (i.e. liquid or in the form of a gel plaster).

The invention has the advantage that with a single collection of blood from the patient it is possible to make a plurality of doses of the composition. The number of doses is comprised between 1 and 4, preferably 2, for both the liquid compositions and the compositions in gel form.

The device (1) and the method enables use of volumes of whole blood comprised between 10 and 200 mL.

The device (1) defines and limits a closed and sterile environment, in which the composition comprising PRP can be manufactured.

Further, the device (1) enables making the production process of the composition comprising PRP completely automatic, with the manual intervention of the operator for the gelation.

The device (1) performs a substantially vertical centrifugation of the collected blood.

The device (1) carries out a double centrifugation, both with controlled deceleration. The controlled deceleration of the first step enables facilitating the separation of the red blood cells and the white blood cells from the plasma, while avoiding remixing. In general, the controlled deceleration further enables reducing the stress the platelets are subjected to. Therefore, by piloting the deceleration gradually and thus avoiding sharp changes of velocity or acceleration, it is possible to avoid the formation of undesired vortices in the separation container (3) and the consequent remixing of the components.

The polymeric scaffold, in particular comprising polylactic acid, as a function of its thickness, shape and geometry, enables realising a support for the composition comprising PRP which is able to support the composition without loss of material (i.e. without dripping), with a good degree of rigidity, but also able to make the plaster flexible and adaptable to the anatomical regions on which it is to be applied. Further, the presence of the scaffold enables suturing the composition in gel form on the skin surrounding the wound. Further, as the scaffold is made using a material that is naturally antimicrobial, the risk of infections is reduced.

Further, the scaffold enables creation of a functional structure for migration, integration and growth of the cells which are to reform the tissue of the lesion.

The polymeric sponge, if present, according to its composition (for example alginate, gelatin, collagen or chitosan), enables absorption of the whole quantity of liquid PRP, facilitation of the gelation of the PRP, and making the composition in gel form more flexible and adaptable to the shape of the skin lesion.

The composition in gel form can be made with different dimensions, and thus enable adapting the dimension of the composition to the dimension of the wound. Compositions can be made in gel form having, for example, a diameter or a side comprised between 1 and 20 cm, preferably between 3 and 9 cm.

The housing (not illustrated) has the advantage of enabling elimination of substantially a majority of the air from the gelation container (21), preventing the formation of bubbles on the surface or internally of the gel plaster.

Therefore it is possible to obtain a composition in gel form with excellent mechanical characteristics. Further, as the housing is provided with the unit for heating the gelation container (21), for example to 37°, the gelation of the composition itself is facilitated and made more rapid.

The device (1) comprising the gelation container (21) enables maintaining a closed and sterile microenvironment suitable for the gelation of the composition comprising PRP. As the gelation container (21) is closed, the incubating temperature (TI) internally thereof is maintained practically constant and, further, the gel composition is kept sterile up until the opening of the container (21). Further, the gelation container (21) being preferably made of PVC, the composition in gel form does not adhere to the walls thereof. In fact, with respect to other materials PVC has shown that during gelation of the composition does not tend to adhere to plastic surfaces (rather than polystyrene, polycarbonate, polyurethane).

Therefore, the composition in gel form is easily removable from the PVC without loss of biological material, loss of consistency, damage or alteration of the composition itself.

The composition comprising PRP obtained with the device (1) and the relative realisation method is of a higher quality from both the point of view of the functionality and of the effectiveness of the composition itself.

Further, the compositions have a high percentage di platelets retrieved from the whole blood and conserved in the final product, as well as a factor of concentration that is high and comprised at least between 4 and 7.

In a relative embodiment the method to manufacture a composition comprising platelet-rich plasma (PRP) according to the invention comprises a first centrifugation step of the whole blood in a centrifugation unit (2) so as to separate a plasma comprising platelets. The method is characterised in that the first centrifugation step comprises a first sub-step of acceleration having a preset duration (TA1) at a preset acceleration value (A1) to obtain sediment of red blood cells from whole blood; and a subsequent first deceleration sub-step with a deceleration value (D1) preset and comprised between 0.0009 and 0.5 rad/s² with a preset deceleration duration (TD1) of greater than 2 minutes, preferably less than 50 minutes. In this matter, see the experimental data included in the following, indicating that, according to the method a great percentage of platelets can be retrieved given a same collection of blood carried out.

It is preferable for the method to further comprise a second centrifugation step of the plasma comprising platelets so as to separate it into a pellet of platelets and a platelet-poor plasma, the second centrifugation step being subdivided into a second sub-step of acceleration having a preset duration (TA2) and a preset acceleration value (A2) and a second sub-step of deceleration having a preset duration (TD2) with a with a preset deceleration value (D2). An embodiment of the method to manufacture a composition comprising platelet-rich plasma (PRP) is preferable, in particular in which the first step of centrifugation is carried out with a preset deceleration value (D1) comprised between 0.0009 and 0.5 rad/s² with a preset deceleration duration (TD1) of greater than 2 minutes, preferably less than 50 minutes, comprising:

a step of supplying, preferably automatic, of the whole blood to the centrifugation unit (2);

a first centrifugation step of the whole blood so as to separate a plasma comprising platelets from the waste materials;

a step of intermediate storage, preferably automatic, of the plasma comprising platelets;

a step of cleaning, preferably automatic, of the centrifugation unit (2), in particular of the container (3) housed in the centrifugation unit as previously indicated;

a second centrifugation step of the plasma comprising platelets so as to separate it into a pellet of platelets and a platelet-poor plasma;

a step of resuspension of the pellet of platelets, in at least a part of platelet-poor plasma at the end of which a platelet-rich plasma (PRP) is obtained;

wherein each centrifugation step is subdivided into a sub-step of acceleration having a preset duration (TA1, TA2) and/or carried out at a preset acceleration value (A1, A2) and a sub-step of deceleration having a preset duration (TD1, TD2) with a with a preset deceleration value (D1, D2).

In this method, the value (A1) of the first acceleration is preferably lower than the value (A2) of the second acceleration, the first acceleration duration (TA1) is shorter or longer than the second acceleration duration (TA2) and the second deceleration duration (TD2) is shorter than the first deceleration duration (TD1).

The method of the invention advantageously comprises a step of resuspension of the pellet of platelets which comprises the automatic insertion in the centrifugation unit (2) of a predefined part of the platelet-free plasma and a successive plurality of further centrifugation steps having a duration comprised between 0.2 seconds and 1 minute.

The method preferably comprises a further sub-step of collecting, preferably automatically, the composition comprising the platelet-rich plasma (PRP) in the substantially liquid state in a collecting container (19).

Alternatively the proposed method can comprise further sub-steps of: supplying, preferably automatically, the platelet-rich plasma (PRP) to a gelation container (21) in which a polymeric scaffold and preferably a polymeric sponge are arranged; heating the gelation container (21) to an incubating temperature (TI) comprised between 35 and 42° C., in particular about 37° C.; and supplying a gelling fluid to the gelation container (21), so as to obtain the composition in gel form.

The apparatus (100) for centrifugation of the invention with which the method can be actuated as claimed in claim 1 comprises:
a centrifugation station in which a separation container (3) is housable to be subjected to centrifugation in order to obtain a centrifugation unit (2);
a electronic control unit (ECU) configured to be able to activate the centrifugation unit (2) in a first centrifugation step with a preset acceleration value (A1) and a preset duration of acceleration (TA1) to obtain sediment of red blood cells from whole blood;

wherein the electronic control unit (ECU) is configured to be able to activate, at the end of the first centrifugation step, the centrifugation unit (2) in a first step of deceleration with a with a preset deceleration value (D1) comprised between 0.0009 and 0.5 rad/s² with a preset deceleration duration (TD1) of greater than 2 minutes, preferably less than 50 minutes.

The apparatus (100) for centrifugation preferably further comprises:
a first and a second peristaltic pump (7, 11) configured to be engageable with conduits in order to pump a relative fluid present internally of the conduits;
three tube-clamp valves (34), each of which is predisposed to engage with a transversal section of a corresponding hydraulic conduit (14, 15, 16) to enable closing and opening the corresponding hydraulic conduit (14, 15, 16);
a plurality of sensors (18) wherein each sensor (18A, 18B, 18C, 18D) of the plurality (18) of sensors comprises a relative recess or engaging means for engaging with a transversal section of a hydraulic conduit, at least partly transparent and is predisposed to transmit, to the electronic control unit (ECU), a datum relative to the quantity of light transmitted or absorbed by the transversal section and wherein the electronic control unit (ECU) is predisposed to be able to compare the relative datum, when transmitted by one of the sensors (18A, 18B, 18C, 18D) with a plurality of reference data relative to: whole blood, red blood cells, plasma and air.

This enables obtaining, by combining the apparatus with a single-use kit according to the invention, a device (1) with which to automate and control various steps of the method of the invention, in particular by using a closed hydraulic circuit which conserves the sterility of the PRP obtained.

In a preferred aspect of the invention the apparatus (100) further comprises a housing in which a gelation container (21) is housable and wherein, optionally, the housing comprises at least an element selected from among:
a heating unit for heating, in use, at least a wall (23) or (24) of the gelation container (21) at least at an incubating temperature (TI) comprised between 35° and 42° C.;
compression means for compressing the gelation container (21) when housed, comprising: elastic return means, a plate and a support which are connected to one another by the elastic return means.

A preferred embodiment of the device (1) to manufacture a composition comprising platelet-rich plasma (PRP) comprises:
a first collection container (4) of the whole blood;
a second container (5) for intermediate storage of a plasma comprising platelets or a platelet-poor plasma, separated from the collected whole blood;

optionally, a container (6) comprising a cleaning liquid;

a collecting unit (17) of the composition comprising the platelet-rich plasma (PRP); a centrifugation unit (2) of the whole blood, comprising a separation container (3) which is set in rotation in two successive centrifugation cycles in order to separate the components of the whole blood so as to obtain the platelet-rich plasma (PRP);

a first pump (7) configured to supply the whole blood from the first container (4) to the centrifugation unit (2) and to supply the waste components obtained at the end of the two centrifugation cycles from the centrifugation unit (2) to the first container (4);

a second pump (11) configured to supply the plasma comprising platelets from the centrifugation unit (2) to the second container (5) at the end of the first centrifugation cycle, and vice versa; and to supply the composition comprising the platelet-rich plasma (PRP) towards the collecting unit (17); and preferably also to supply the waste platelet-poor plasma from the centrifugation unit (2) to the second container (5) at the end of the second centrifugation cycle; a electronic control unit (ECU) configured to activate the centrifugation unit (2) with a preset acceleration value (A1, A2) and/or a preset acceleration duration (TA1, TA2);

wherein the electronic control unit (ECU) is configured to activate the centrifugation unit (2) and/or a preset deceleration value and/or and/or a preset deceleration duration (TD1, TD2) at the end of each acceleration duration (TA1, TA2).

The device (1) can advantageously comprise a plurality of sensors (18, 18A, 18B, 18C, 18D) configured to detect at least a characteristic from among: the infeed flow rate of the first pump (7) or the second pump (11), the presence of the conduits (8, 12, 14, 15, 16), the presence of bubbles in conduits (8, 12, 14, 15, 16), the presence of the fluid to be supplied to the relative conduit (8, 12, 14, 15, 16) and/or the change in turbidity of the fluid to be supplied into the relative conduit (8, 12, 14, 15, 16). In a preferred embodiment of the device (1), the relative collecting unit (17) can be configured to house a collecting container (19) of the composition, in particular a syringe.

The device (1) can advantageously comprise a gelation container (21) connectable to the collecting unit (17) and configured to internally house a polymeric scaffold, in particular polylactic acid, and, optionally a polymeric sponge; the gelation container (21) comprises at least a lower wall (23) and/or an upper wall (24) which is elastically deformable. In this case the gelation container (21) can preferably comprise a first connecting means (22) for connecting the container (21) to the collecting unit (17) and a second connecting means (26) for connecting a conveying means of a gelling fluid, in particular a syringe comprising calcium gluconate.

The device (1) of the invention can advantageously comprise: a housing for the gelation container (21) provided with a first plate and a support which are connected to one another, preferably by elastic return means so as to exert a compression force on the gelation container (21) interposed between them.

In a preferred embodiment, the device (1) can comprise a heating unit for heating, in use, at least a surface (23, 24) of the gelation container (21).

Also advantageous is a kit for a device (1) to manufacture a composition comprising platelet-rich plasma (PRP) comprising at least an element selected from among: a collection container (4); a container (6) comprising a cleaning liquid; an intermediate storage container (5); a plurality of conduits (8, 12, 14, 15, 16), in particular suitable for being inserted in peristaltic pumps in order to pump a relative fluid present internally of the conduits or in tube-clamp valves (34); a multi-way connector; and a separation container (3). In order to be inserted in a peristaltic pump (7, 11) or in a tube-clamp valve (34), it is sufficient for the respective conduits (8, 12, 14, 15, 16) to have at least a relative transversal section, compressible and insertable in appropriate housings in the peristaltic pump (7, 11) and in the tube-clamp valve (34). In order for a sensor (18A, 18B, 18C, 18D) to transmit to the electronic control unit (ECU) a datum relative to the quantity of transmitted or absorbed light by the respective conduits (8, 12) the conduits must have a relative transversal section, at least partly transparent. Note that even where it is not specified, the term “conduits” refers to hydraulic pipes.

The kit preferably comprises a polymeric scaffold and preferably a polymeric sponge.

In a particularly preferred embodiment of the invention, the kit comprises:
a separation container (3) which comprises a relative inlet, a relative fixed wall and a movable wall which define a relative variable internal volume for containing a liquid, wherein the movable wall is movable with respect to the fixed wall to vary the internal volume;
a collection container (4);
a container (5) for intermediate storage;
a container (6) containing a cleaning liquid;
at least a first, a second, a third, a fourth and a fifth conduit (8, 12, 14, 15, 16), each of which comprises at least a relative transversal section suitable for being inserted in a peristaltic pump in order to pump a relative fluid present internally of the conduits (8, 12) and/or so as to be engaged with a tube-clamp valve (34) to enable closing and opening the corresponding hydraulic conduit (14, 15, 16), wherein the first, second, third, fourth and fifth conduit (8, 12, 14, 15, 16) have a first and a second respective end, wherein the first end of the first, third and fourth conduit (8, 14, 15) is connectable, respectively, to the collection container (4); to the container (5) for intermediate storage and to the container (6) containing a cleaning liquid; and wherein, optionally, the first and second conduit (8, 12) comprise at least a further relative transversal section, at least partly transparent;
a four-way connector (13), wherein each relative different single way of the connector is hydraulically connectable, respectively, to the second end of the second, third, fourth and fifth conduit (12, 14, 15, 16);
a collection interface (17) hydraulically connectable to the first end of the fifth conduit (16);
a connecting element (EC) comprising a three-way connector, wherein each relative different single way of the connector is hydraulically connectable, respectively to the first end of the second conduit (12), to the second end of the first conduit (8) and to the inlet of the separation container (3); and, optionally, means for reducing friction, preferably constituted by a rotary joint, arranged at one of the ways to enable hydraulic connection of the way to the inlet of the separation container (3) even when the separation container is subjected to centrifugation.

The kit advantageously further comprises a collecting unit (20) selected from among:
a first collecting unit (20) comprising a connecting means (22) hydraulically connectable to the interface (17) and a gelation container (21) comprising at least a lower wall (23) and/or an upper wall (24) which is elastically deformable, and configured to internally house a polymeric scaffold, in particular made of polylactic acid, and preferably a polymeric sponge;
a second collecting unit (20) comprising a connecting means (22) hydraulically connectable to the interface (17) and at least a collecting container (19), and, optionally, at least a multi-way hydraulic branch point in which each way is connectable to a single collecting container (19) provided. In this case, three containers and one X-shaped hydraulic branch point can be included as visible in FIG. 3a. The first collecting unit (20) preferably comprises the polylactic polymeric scaffold, and/or the polymeric sponge housed internally of the gelation container.

A composition is preferably, comprising platelet-rich plasma (PRP) manufactured with a method according to the invention, advantageously when the composition is in a gel form and comprises: a polymeric scaffold, in particular made of polylactic acid, having a grid structure, with a thickness comprised between 5 and 500 μm; and preferably a polymeric sponge manufactured in particular with a material selected from among: alginate, gelatin, collagen and/or chitosan, and having a thickness comprised between 1 mm and 10 mm, preferably between 1 mm and 3 mm.

The composition for use as a medication is preferred, in particular for the treatment of skin lesions and/or of osteochondral or joint pathologies, in particular the use of the composition as a medication is preferred, in particular in the treatment of skin lesions and/or in osteochondral or joint pathologies and/or the use of the composition for preparation of a medication for the treatment of skin lesions and/or for osteochondral or joint pathologies.

The technical expert in the sector will clearly see, in the light of the present patent application and relative figures, how to connect the various components of the kit (10) of the invention to one another, how to connect the elements of the apparatus (100) for centrifugation according to the invention to obtain the device (1) according to the invention and how to activate the tube-clamp valves (34) and the pumps (7 and 11) in order to actuate the various embodiments of the method of the invention.

### Experimental Data

The acceleration data is reported as Relative Centrifugal Force (RCF) having as measuring unit the unit of acceleration g. As a vertical single station centrifugation apparatus is used having a rotor radius that is the same as the radius of the separation container (3) of 20 mm Conversion formula from Rotations per minute (RPM) to g or to RCF:

RCF=1.12×Rotor radius in mm×(RPM/1000)².

### EXAMPLES

### Comparative Example 1

A sample of peripheral venous whole blood was taken, divided into aliquots of 50 ml, and subjected to the first centrifugation with a common bench centrifuge following the protocol described in Daniel Tzu-BiShih et al. “Preparation, quality criteria, and properties of human blood platelet lysate supplements for ex vivo stem cell expansion” New Biotechnology” Volume 32, Issue 1, 25 Jan. 2015, Pages 199-211, being
1st Centrifugation at 1000 g×10 minutes
2nd Centrifugation at 3000 g×5 minutes

In this process the normal deceleration of the bench centrifuge was used, which was timed at 45 seconds.

### Example 1

The second aliquot was used to carry out a centrifugation test using the apparatus for centrifugation according to the invention, following the protocol below:
1st Centrifugation with an acceleration value (A1) of 200 g and a duration (TA1) of 3 minutes, with a deceleration value (D1) comprised between 0.0009 and 0.5 rad/s2 and a deceleration duration (TD1) of 14 minutes,
2nd Centrifugation with an acceleration value (A2) of 750 g and a duration (TA2) of 5 minutes, with a deceleration duration (TD1) of 5 minutes.

The preparation of the PRP was carried out following the same procedures for both tests: the pellet of platelets was resolubilised in a volume of plasma of 10% of the volume of whole blood collected, of 5 ml. To assess the effectiveness of the methods and the devices used, at the end of each centrifugation step (first and second), samples of plasma and PRP were collected to carry out the count of platelets, white blood cells and red blood cells. The counts of the samples have been carried out with the ABBOT DIAGNOSTICS Model CELL DYN 3500 Plus blood analyser.

The quantities retrieved for each cell population were compared with those obtained from the count carried out on the original sample of whole blood.

The results of the 1st centrifugation were reported in the following table:

TABLE 1
First centrifugation (obtaining plasma)

| | Duration of the | % of recovery | % of RBC |
|---|---|---|---|
| Method | deceleration | of platelets | recovery |
| Comparative | 45 seconds | 63% | <1% |
| example 1 | | | |
| Example 1 | 14 minutes | 75% | <1% |

The apparatus for centrifugation and the method of the invention enable retrieval of a greater quantity of platelets in the plasma from the whole blood.

The results of the 2nd centrifugation and therefore the parameters relative to the PRP obtained in the two examples, are reported in the following table:

TABLE 2
Second centrifugation (obtaining PRP)

| | % of recovery | Platelets | % of RBC |
|---|---|---|---|
| Method | of platelets | concentration factor | recovery |
| Comparative | 52% | 5.2 | <1% |
| example 1 | | | |
| Example 1 | 71% | 7.1 | <1% |

The final recovery of the platelets in the final preparation (PRP) is greater when using the apparatus for centrifugation and the method according to the invention. The factor of platelet concentration obtained (defined as the number of times in which the platelet concentration is incremented in the PRP with respect to whole blood), is significantly greater with the apparatus and the method of the invention. A greater factor of concentration is translated into a greater effectiveness of the preparation.

Further comparative analyses were made, with aliquots of the same blood collection in order to evaluate the effectiveness of the processes of deceleration of the centrifuges today available using the acceleration values and durations indicated in the above-mentioned document by Daniel Tzu-BiShih et al.. Thus using bench centrifuges having different types of rapid deceleration (Comparative example 1V), medium deceleration (Comparative example 1M), and slow deceleration (Comparative example 1M), PRP preparations were carried out following the protocol of the above-mentioned document authored by Daniel Tzu-BiShih et al., obtaining the results reported in the table below, in which we also report the results obtained with the method and the apparatus according to the invention.

TABLE 3
First centrifugation (obtaining plasma)

| | Duration of the | % of recovery | % of RBC |
|---|---|---|---|
| Method | deceleration | of platelets | recovery |
| Example 1 | 14 minutes | 75% | <1% |
| Comparative | 32 seconds | 65% | <1% |
| example 1V | | | |
| Comparative | 1 minute and 13 | 54% | <1% |
| example 1M | seconds | | |
| Comparative | 1 minute and 32 | 54% | <1% |
| example 1L | seconds | | |

TABLE 4
Second centrifugation (obtaining PRP)

| | % of recovery | platelets | % of RBC |
|---|---|---|---|
| Method | of platelets | concentration factor | recovery |
| Example 1 | 71% | 7.1 | <1% |
| Comparative | 55% | 5.5 | <1% |
| example 1V | | | |
| Comparative | 41% | 4.1 | <1% |
| example 1M | | | |
| Comparative | 43% | 4.3 | <1% |
| example 1L | | | |

From the total of the comparative tests carried out, the outcome of which is summarised in the following table 5, it is clear that, according to the invention a percentage of recovery of platelets can be obtained that is significantly greater than what is obtainable according to the above-cited document by Daniel Tzu-BiShih et al. Independently of the type of bench centrifuge of known type at present available. This enables increasing the performance of platelet recovery, and also enables, given a same aliquot of whole blood collected, a more efficient PRP in the relative therapeutic use.

TABLE 5

| Method | % of recovery of platelets |
|---|---|
| Example 1 | 71% |
| Comparative example 1 | 52% |
| Comparative example 1V | 55% |
| Comparative example 1M | 41% |
| Comparative example 1I | 43% |

Examples of the combination of the acceleration values A1, A2 of deceleration D1, D2 and relative preset durations TA1, TA2, TD1, TD2.

First centrifugation step:

TABLE 6

| | A1 g | TA1 min | D1 rad/s² | TD1 min |
|---|---|---|---|---|
| Example 1.1 | 100 | 10 | 0.36 | 10 |
| Example 1.2 | 180 | 10 | 0.38 | 13 |
| Example 1.3 | 200 | 3 | 0.37 | 14 |
| Example 1.4 | 500 | 10 | 0.41 | 20 |

Second centrifugation step:

TABLE 7

| | A2 g | TA2 min | D2 rad/s² | TD2 min |
|---|---|---|---|---|
| Example 2.1 | 500 | 3 | 2.75 | 3 |
| Example 2.2 | 750 | 5 | 2 | 5 |
| Example 2.3 | 900 | 5 | 11 | 1 |
| Example 2.4 | 900 | 5 | 1.84 | 6 |

1-21. (canceled)

22. A composition comprising platelet-rich plasma (PRP), wherein the composition is in a gel form comprising:
a polymeric scaffold, made of polylactic acid, having a grid structure, with a thickness between 5 and 500 μm; and
a polymeric sponge manufactured with a material selected from the group consisting of alginate, gelatin, collagen and chitosan and combinations thereof, and having a thickness between 1 mm and 10 mm.

23. The composition according to claim 22 for use as a medication.

24. The composition for use according to claim 23 for the treatment of skin lesions and/or for osteochondral or joint pathologies.

25. The composition according to claim 22, wherein the polymeric sponge has a thickness between 1 mm and 3 mm.

26. A collecting unit comprising:
a first connector;
a gelation container having a plurality of walls, wherein at least one of the walls is elastically deformable, so as to deform under the action of a compression force exerted thereon, wherein the first connector is coupled with the gelation container;
a second connector for connecting a source of a gelling fluid to the gelation container;
an air filter in communication with the gelation container;
a polymeric scaffold manufactured with a biopolymer and arranged in the gelation container; and
a polymeric sponge disposed in contact with the polymeric scaffold.

27. The collecting unit according to claim 26, wherein the polymeric scaffold is manufactured in polylactic acid, and has a grid structure, with a thickness comprised between 5 and 500 μm; and
the polymeric sponge is made of a material selected from the group consisting of alginate, chitosan, gelatin and collagen and combinations thereof and has a thickness between 1 mm and 10 mm.

28. The collecting unit according to claim 27, wherein the polymeric sponge has a thickness between 1 mm and 3 mm.

29. The collecting unit according to claim 26, wherein the first connector is a Luer connector, the second connector is needle-less, and the biopolymer is polylactic acid.

30. A gelation container connectable to a collecting unit and configured to internally house a polylactic acid scaffold and a polymeric sponge.
